(19) United States
(12) Patent Application Publication
(10) Pub. No.: US 2003/0190377 A1
(43) Pub. Date: Oct. 9, 2003

(54) Novel use of the extract of processed Panax genus plant and saponin compound isolated therefrom

(75) Inventors: Dong-Hyun Kim, Seoul (KR); Eun-Ah Bae, Seoul (KR); Myung-Joo Han, Seoul (KR); Min-Kyung Choo, Seoul (KR); Eun-Kyung Park, Seoul (KR); Jeong-Hill Park, Seoul (KR)

Correspondence Address: FOLEY AND LARDNER, SUITE 500, 3000 K STREET NW, WASHINGTON, DC, 20007, US

(73) Assignee: Ginseng Science Inc.

(21) Appl. No.: 10/345,208

(22) Filed: Jan. 16, 2003

(30) Foreign Application Priority Data
Apr. 8, 2002 (KR) 10-2002-0018843
Dec. 28, 2002 (KR) 10-2002-0085955

(51) Int. Cl. A61K 35/78; A61K 31/366

(52) U.S. Cl. 424/728; 514/460

(57) ABSTRACT

The present invention relates to novel use of the extract of processed Panax genus having anti-Helicobacter pylori activity. More particularly, the present invention relates to a processed Panax genus extract with enhanced pharmacological effects due to subsequent treatment i.e., acid-treatment or heat-treatment of a Panax genus plants and bio-converting treatment such as lactic acid bacterial fermenting and intestinal bacterial fermenting process so as to make a ratio of ginsenoside (Rk₂+Rh₃+protopanaxadiol+20-dehydroprotopanaxadiol) to (Rg₃+Rg₅+Rk₁) of above 0.1. The extract of processed Panax genus plant in the present invention has inhibitory effect for Helicobacter pylori bacteria and H⁺/K⁺-ATPase enzyme and, therefore, it is useful in the prevention or treatment of gastrointestinal diseases caused by abnormal proliferation of Helicobacter pylori such as gastritis, gastric ulcer, duodenal ulcer and gastric cancer.

The present invention relates to novel use of the extract of processed Panax genus having anti-Helicobacter pylori activity. More particularly, the present invention relates to a processed Panax genus extract with enhanced pharmacological effects due to subsequent treatment i.e., acid-treatment or heat-treatment of a Panax genus plants and bio-converting treatment such as lactic acid bacterial fermenting and intestinal bacterial fermenting process so as to make a ratio of ginsenoside (Rk₂+Rh₃+protopanaxadiol+20-dehydroprotopanaxadiol) to (Rg₃+Rg₅+Rk₁) of above 0.1. The extract of processed Panax genus plant in the present invention has inhibitory effect for Helicobacter pylori bacteria and H⁺/K⁺-ATPase enzyme and, therefore, it is useful in the prevention or treatment of gastrointestinal diseases caused by abnormal proliferation of Helicobacter pylori such as gastritis, gastric ulcer, duodenal ulcer and gastric cancer.

### DESCRIPTION

1. FIELD OF THE INVENTION

The present invention relates to novel use of an extract of processed Panax genus plant and saponin compounds isolated therefrom for treat gastrointestinal disease caused by abnormal proliferation of Helicobacter pylori bacteria in human or mammal. More particularly, the present invention relates to novel use of processed ginseng product with enhanced pharmacological effects due to subsequent treatment i.e., acid-treatment or heat-treatment of a Panax genus plants and bio-converting treatment such as lactic fermenting and intestinal-bacterial fermenting process so as to make a ratio of ginsenoside (Rk₂+Rh₃+protopanaxadiol+20-dehydroprotopanaxadiol) to (Rg₃+Rg₅+Rk₁) of above 0.1.

2. BACKGROUND OF THE INVENTION

It is known that there are many genus of Panax genus plants belonged to Araliaceae, for example, Panax ginseng distributed or cultivated in far-eastern Asia region, Panax quinquefolia in America and Canada, Panax notoginseng in China, Panax trifolia in eastern region of north America, Panax japonica in Japan, China and Nepal, Panax pseudoginseng in Nepal, Panax vietnamensis in Vietnam, Panax elegatior, Panax wangianus and Panax bipinratifidus etc.

Hitherto, a ginseng has been widely known as a representative nutritive tonic agent. Recently, various scientific studies on the chemical constituents and pharmacological effects of the ginseng have been reported so that the secret pharmacological effects are paid attention with modern scientific approaches. Until now, it has been known that the ginseng has various pharmacological effects such as prevention of aging, anti-arteriosclerosis, treatment of hyperlipidemia, treatment of hepatic insufficiency, improvement of liver function, protection of radiation injury, immune enhancement, improvement of cerebral function, anti-thrombotic, anti-stress, anti-diabetic, anti-hypertensive, anti-tumor effects, etc.

It has been known that the main constituent of Panax genus plant is dammarane-skeleton type saponin. Ginsenosides Rb₁, Rb₂, Rc, Rd, Rg₁, and Re are the main saponins in Panax ginseng. Their biological activities are different from each other in accordance with their chemical structures.

There have been many attempts to modify the structure of the saponins to increase their pharmacological potency through processing.

Korean Patent Publication No. 10-1996-017670 issued on May. 23, 1996, discloses a process for preparing a processed ginseng prepared by subjecting hot temperature treatment containing high contents of ginsenoside Rg₃ and Rg₅ so as to obtaining processed ginseng having improved potency differing from original form of ginseng.

Korean Patent Publication No. 10-1996-004217 issued on Feb. 22, 1996, discloses a process for the production of saponin metabolites such as compound K from ginseng saponins using intestinal-bacteria.

However, there have been no disclosure or suggestion about a process for preparing processed Panax genus plant prepared by serial treatment comprising acid or heat treatment or their combination thereof, and subsequent fermentation treatment with lactic-acid bacteria or intestinal-bacteria so as to chemically change their saponin components resulting in a ratio of ginsenoside (Rk₂+Rh₃+protopanaxadiol+20-dehydroprotopanaxadiol) to (Rg₃+Rg₅+Rk₁) of above 0.1.

The inventors of the present invention have intensively carried out the scientific investigation concerning chemical constituents and pharmacological effects of a ginseng, in particular a processing method of a ginseng and physiological activity of the processed ginseng. As a result of the investigation, the inventors have discovered that the serial treatment comprising acid or heat treatment or their combination thereof, and subsequent fermentation treatment with lactic-acid bacteria or intestinal-bacteria, the extract of Panax plant shows substantially enhanced pharmacological effects, especially, anti-helicobacter activity and they have finally completed the present invention.

### SUMMARY OF THE INVENTION

Accordingly, it is an object of the present invention to provide a use of Panax genus plant or the extract thereof comprising a ratio of ginsenoside (Rk₂+Rh₃+protopanaxadiol+20-dehydroprotopanaxadiol) to (Rg₃+Rg₅+Rk₁) of above 0.1, in the manufacture of a medicament for the prevention or treatment of gastro-intestinal disease.

And, another object of the present invention is to provide a use of panaxytriol, panaxydol, panaxynol, ginsenosides Rc, Rb₁, Rg₃, Rg₅, Rh₁, Rh₂, protopanaxadiol, protopanaxatriol and the mixture thereof, in the manufacture of a medicament for the prevention or treatment of gastro-intestinal disease.

And, another object of the present invention is to provide a use of Panax genus plant or the extract thereof obtained by the steps essentially comprising acid or heat treating or their combination thereof the plant material belonged to Panax genus, and subsequent fermentation treating with lactic-acid bacteria or intestinal-bacteria.

An additional object of the present invention is to provide a method for treating or preventing gastro intestinal disease in a mammal comprising administrating to said mammal an effective amount of above extract or the saponin compounds isolated therefrom, together with a pharmaceutically acceptable carrier thereof.

### DETAILED DESCRIPTION

In accordance with the present invention, the present invention provides a pharmaceutical composition comprising processed Panax plant or the extract thereof wherein the ratio of ginsenoside (Rk₂+Rh₃+protopanaxadiol+20-dehydroprotopanaxadiol) to (Rg₃+Rg₅+Rk₁) of above 0.1, preferably, 0.2, more preferably 0.5, as an active ingredient in an amount effective to treat or prevent human or mammal gastro-intestinal diseases caused by abnormal proliferation of Helicobacter pylori, together with a pharmaceutically acceptable carrier.

The present invention also provides a pharmaceutical composition comprising the extract of Panax genus plant obtained by the steps essentially comprising acid or heat treating or their combination thereof the plant material belonged to Panax genus, and subsequent fermentation treating with lactic-acid bacteria or intestinal-bacteria, as an active ingredient in an amount effective to treat or prevent human or mammal gastro-intestinal diseases caused by abnormal proliferation of Helicobacter pylori, together with a pharmaceutically acceptable carrier.

The present invention provides a use of Panax genus plant or the extract thereof comprising a ratio of ginsenoside (Rk₂+Rh₃+protopanaxadiol+20-dehydroprotopanaxadiol) to (Rg₃+Rg₅+Rk₁) of above 0.1, preferably, above 0.2, more preferably above 0.5, wherein said protopanaxadiol (PPD) and 20-dehydroprotopanaxadiol (DHPPD) comprise their isomers, i.e., (20S) PPD, (20R) PPD and 20(21)-DHPPD, 20(22)-DHPPD to prevent or treat gastro-intestinal disease.

The present invention also provides a use of processed Panax genus plant or the extract thereof obtained by the steps essentially comprising acid or heat treating or their combination thereof the plant material belonged to Panax genus, and subsequent fermentation treating with lactic-acid bacteria or intestinal-bacteria to prevent or treat gastro-intestinal disease.

Additionally, the present invention also provide a method for treating or preventing gastro-intestinal disease in a mammal comprising administrating to said mammal an effective amount of Panax genus plant, above extract or the saponin compounds isolated therefrom, together with a pharmaceutically acceptable carrier thereof.

Above described plant or extract and the saponin compounds therefrom can be prepared by following steps:

1. 1^{st} step:

1^{st} step is to subject following acid or heat treatment step or the combinations thereof to plant material as follows;

(1) Acid treatment step

Specifically, at the 1^{st} step, dried plant material of Panax genus, for examples, the root of Panax ginseng, is subjected to following acid treatment; for example, about 1 to 50 times, preferably 5 to 20 times of 0.01 to 50%, preferably, 0.1 to 10% acidic component, preferably, acetic acid, citric acid,, lactic acid or acid-containing food such as the fruit of Schisandra chinensis, is added to the plant material and then is subjected to incubation at a temperature ranging from 20 to 80° C., preferably 40 to 70° C. for a period ranging from 1 to 48 hrs, preferably, 3 to 12 hrs. Organic solvent such as methanol, ethanol, propanol, butanol, ether, ethyl acetate is added thereto and then subjected to extraction to obtain organic solvent soluble extract; the extract is neutralized with base finally to obtain the extract of chemically processed Panax genus.

(2) Heat treatment step

As another initial step to obtain the present invention, heat treatment process can be employed, i.e., dried plant material or its extract of Panax genus is subjected to following heat treatment; for example, the plant material or its extract is treated at a temperature ranging from 110 to 180° C., preferably, 120 to 140° C. for a period ranging from 0.5 to 20 hours, preferably 2 to 5 hours. The heating time varies depending on the heating temperature. The lower heating temperature requires the longer heating time. The heating procedure may be carried out by using a hot air, steam, nitrogen, helium, carbon dioxide, oxygen or mixed gas thereof. In order to increase the efficiency, the heating process may be preferably performed in an airtight container such as autoclave. Alternatively, a small amount of water may be added to the container; otherwise, the ginseng may be preferably soaked in water and then heated in a closed container.

The ginseng thus processed may be dried at a lower temperature than the heating temperature of the processing procedure, i.e., a normal temperature to 80° C. by a known manner to obtain a dried processed ginseng, or it may be further processed to obtain a powdered ginseng, if necessary.

Alternatively, the processed ginseng may be extracted using a known manner to obtain a processed ginseng extract. Specifically, the processed ginseng is extracted by using a solvent, and then the solvent is removed in vacuo or in freeze-drier to obtain a processed ginseng extract as dried powders.

The solvent which may be employed herein includes a water, lower alcohol such a methanol, ethanol, etc., lower ketone such as acetone, methylethylketone, etc., supercritical fluid or mixed solvent thereof.

The plant material which may be employed includes, but are limited to, Panax genus plant itself such as a fresh ginseng, a white ginseng and red ginseng, a fine root of ginseng or ginseng leaves or extracts thereof, which can be used as it is, finely divided or powdered, processed product thereof and their by-product which comprise dammarane type saponin, preferably, the root, stem, petal, leaf, fruit of Panax ginseng, Panax quinquefolia, Panax notoginseng, Panax trifolia, Panax japonica, Panaxpseudo ginseng, Panax vietnamensis, Panax elegatior, Panax wangianus, Panax bipinratifidus and Panax angustifolium and their tissue cultivates and the extract thereof.

Above (1) and (2) processes can be subjected to plant material respectively or in a combination manner prior to following 2^{nd} step.

2. 2^{nd} step: fermentation step

The extract obtained from 1^{st} step is subsequently subject to following bioconversion process such as fermentation with lactic acid or intestinal-bacteria as follows:

For example, lactic acid bacteria or intestinal-bacteria is added to the extract obtained from 1^{st} step and incubated at a temperature ranging from 20 to 50° C., preferably, 25 to 40° C. for a period ranging from 8 hours to 8 days, preferably 24 hours to 3 days to obtain extract fermented with bacteria.

The incubation time varies depending on the genus of used bacteria.

The lactic acid bacteria which may be employed includes any one which can metabolize ginsenoside Rg₃, Rg₅ and Rk₁, to ginsenoside Rh₂, Rh₃, Rk₂, protopanaxadiol (PPD) and 20-dehydroprotopanaxadiol (DHPPD), preferably, lactic acid bacteria belonged to Bifidobacterium genus, more preferably, at least one or the mixture thereof selected from the group consisting of Bifidobacterium infantis, Bifidobacterium bifidum, Lactobacillus lactis, Clostridium butyricum, Bifidobacterium K-103, Bifidobacterium K-506, Bifidobacterium K-513, Bifidobacterium K-525, Bifidobacterium KK-1 and Bifidobacterium KK-2 (disclosed in Arch. Pharm. Res., 21, p54-61, 1988).

The intestinal bacteria which may be employed includes any one which can metabolize ginsenoside Rg₃, Rg₅ and Rk₁, to ginsenoside Rh₂, Rh₃, Rk₂, protopanaxadiol (PPD) and 20-dehydroprotopanaxadiol (DHPPD), preferably, intestinal-bacteria belonging to Bacterioides, Fusobacterium and Eubacterium genus, more preferably, at least one or the mixture thereof selected from the group consisting of Bacteriodes JY-6 (disclosed in Biol. Pharm. Bull., 23, pp1481-1485, 2000), Bacteriodes stercoris, Fusobacterium K-60 (disclosed in Biol. Pharm. Bull., bid.), Eubacterium L-8 (disclosed in Biol. Pharm. Bull., bid.).

Further to above described steps, to isolate the saponin fractions or the saponin compounds from the extract obtained from above 2^{nd} step, following process can be adopted.

3. 3^{rd} step: Isolation process

To isolate pharmacologically active fractions or saponin compounds from the extract prepared by 2nd step, water, lower alcohols such as methanol, ethanol, propanol, butanol, ethylacetaie, dichloromethane, chloroform, hexane, ether, or the mixed solvent thereof can be used to extract or isolate the fractions or compounds from the extract obtained from 2^{nd} step as an appropriate solvent.

Additionally, the active ingredient can be extracted or isolated by subjecting special extraction method such as supercritical fluid extraction (SFE) to obtain partially purified saponin fractions and further, silica gel column chromatographic method to isolate individual saponins thereby.

Subsequent to above step, following processes such as drying process by lyophilization, agitation or dilution process can be adopted in addition to the above steps, if necessary.

Following processes can be selected either or both according to the final product forms of the present invention.

4. 4^{th} step: Drying process

(1) Above extract of Panax genus plant obtained in Step 2 or 3, is concentrated in vacuo and then dried by lyophilization or spray drying.

(2) Above extract of Panax genus plant obtained in Step 2 or 3, is centrifuged to remove its impurities and precipitate and the supernatant is concentrated in vacuo and then dried by lyophilization or spray drying.

Through above 1^{st} step to 2^{nd} step processes, saponins such as ginsenoside Rb₁, Rb₂, Rc, Rd etc contained in plant material is transformed into chemically modified ginsenosides such as ginsenoside Rg₃, Rg₅, Rk₁, etc due to acid treatment or heat treatment in step 1 and then the sugar moiety at the position 3 in modified saponins is further degraded to form further modified saponins comprising degraded saponins such as ginsenoside Rk₂, Rh₂, Rh₃, PPD, DHPPD, which make substantially novel extract comprising novel components such as ginsenoside Rk₂, Rh₂, Rh₃, PPD, DHPPD absent or present in a trace amount in a commercial ginseng product.

In particular, the processed ginseng product according to the present invention wherein a ratio of ginsenoside (Rk₂+Rh₃+protopanaxadiol+20-dehydroprotopanaxadiol) to (Rg₃+Rg₅+Rk₁) of above 0.1 shows superior physiological activities as different from the prior processed ginseng product in which ginsenoside components such as Rk₂, Rh₂, Rh₃, PPD and DHPPD are hardly present.

Additionally, the present invention provides pharmaceutical compositions comprising at least one saponin compound or the mixtures thereof selected from the group consisting of panaxytriol, panaxydol, panaxynol, ginsenoside Rc, Rb₁, 20(S)-ginsenoside Rg₃, 20(R)-ginsenoside Rg₃, 20(S)-ginsenoside Rh₂, 20(R)-ginsenoside Rh₂, 20(R)-protopanaxadiol, 20(S)-protopanaxadiol, 20(S)-ginsenoside Rh₁, 20(S)-protopanaxatriol and the mixture thereof, as an active ingredient in an amount effective to treat or prevent human or mammal gastro-intestinal diseases, together with a pharmaceutically acceptable carrier.

Specifically, the present invention also provides a use of saponin compounds comprising at least one saponin compound or the mixtures thereof selected from the group consisting of panaxytriol, panaxydol, panaxynol, ginsenoside Rc, Rb₁, 20(S)-ginsenoside Rg₃, 20(R)-ginsenoside Rg₃, 20(S)-ginsenoside Rh₂, 20(R)-ginsenoside Rh₂, 20(R)-protopanaxadiol, 20(S)-protopanaxadiol, 20(S)-ginsenoside Rh₁, 20(S)-protopanaxatriol and the mixture thereof, to prevent or treat gastro-intestinal disease.

The above-described gastro-intestinal disease comprises all the disease in gastro-intestinal tract caused by abnormal proliferation of Helicobacter pylori such as gastritis, gastric ulcer, duodenal ulcer, gastric cancer and the like.

The present invention also provides a method for treating or preventing human or mammal gastro-intestinal diseases comprising administrating to said mammal an effective amount of above described extract and the saponin compounds isolated therefrom and pharmaceutically acceptable carrier thereof.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 1 to 5 g/Kg by weight/day of the inventive extract or compounds of the present invention. The dose may be administered in single or divided into several times per day. In terms of composition, the complex herbal composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

The present inventors demonstrated that anti-helicobacter effect of present composition is more potent than that of the Panax genus plant extract prepared by conventional method or simple processed method such as sole acid treatment or heat treatment by accomplishing in vitro and in vivo experiment, e.g., anti-Helicobacter pylori activity test, assay of rat stomach H⁺/K⁺-ATPase inhibition test, therefore, it is confirmed that above described composition is very useful in the prevention or treatment of gastro-intestinal disease.

Accordingly, it is another object of the present invention to provide a health care food comprising above described extract prepared by above processes and a sitologically acceptable additive to prevent gastro-intestinal disease.

Above described composition therein can be added to food, additive or beverage for prevention of gastro-intestinal disease. For the purpose of preventing gastro-intestinal disease, wherein, the amount of above described extract or compound in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described extract or compound as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Examples of addable food comprising aforementioned extract therein are various food, beverage, gum, vitamin complex, health improving food and the like.

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

### Comparative Example 1

Preparation of the Extract of Non-Processed Panax Genus Plant

60% ethanol(v/v %) was added to each air-dried and sliced 20 g of Panax ginseng root, Panax quinquefolia root and Panax notoginseng root and refluxed for three hours and concentrated in vacuo to obtain 4.5, 4.0 and 3.7 g of the extract of Panax ginseng root, Panax quinquefolia root and Panax notoginseng root respectively.

### Comparative Example 2

Preparation of the Extract of Acid Treatment Panax Genus Plant

1000 ml of water containing 0.1% lactic acid (v/v %) was added to each air-dried and sliced 20 g of Panax ginseng root, Panax quinquefolia root and Panax notoginseng root and incubated at 60° C. for 5 hours and the cultivates was subjected to the solvent extraction with butanol to obtain 2.5, 2.8 and 3.2 g of acid treated extract of Panax ginseng root, Panax quinquefolia root and Panax notoginseng root respectively.

### Comparative Example 2

Preparation of Acid-Treated Extract of Panax Genus Plant

1000 ml of water containing 0.1% lactic acid (v/v %) was added to each air-dried and sliced 20 g of Panax ginseng root, Panax quinquefolia root and Panax notoginseng root and incubated at 60° C. for 5 hours and the cultivates was subjected to the solvent extraction with butanol to obtain 2.5, 2.8 and 3.2 g of acid treated extract of Panax ginseng root, Panax quinquefolia root and Panax notoginseng root respectively.

### Comparative Example 3

Preparation of Heat-Treated Extract of Panax Genus Plant

Air-dried and sliced 100 g of Panax ginseng root, Panax quinquefolia root and Panax notoginseng root was placed into an autoclave and then was heated by steaming at 130° C. for 3 hours. 60% ethanol (v/v %) was added thereto and then refluxed for three hours to obtain 42, 35 and 37 g of heat-treated extract of Panax ginseng root, Panax quinquefolia root and Panax notoginseng root respectively.

### Example 1

Preparation of Processed Extract of Panax Genus Plant

Each heat-treated extract prepared by Comparative Example 3 in an amount equivalent to 1 g of plant material, i.e., Panax ginseng root, Panax quinquefolia root and Panax notoginseng root was dissolved in 20 ml of distilled water. 100 mg of Fusobacterium K-60 (wet weight) was added thereto and then was incubated at 37° C. for 72 hours. The incubates was centrifuged and the supernatant was concentrated and dried to obtain 550, 530 and 430 mg of processed extract of Panax ginseng root, Panax quinquefolia root and Panax notoginseng root respectively.

### Example 2

Preparation of Processed Extract of Panax Genus Plant

Each heat-treated extract prepared by Comparative Example 3 in an amount equivalent to 1 g of plant material, i.e., Panax ginseng root, Panax quinquefolia root and Panax notoginseng root was dissolved in 20 ml of distilled water. 50 mg of Bifidobacterium K-506 (Disclosed in Arch. Pharm. Res., 21, pp54-61, 1988), 50 mg of Bifidobacterium K-103 (Disclosed in Arch. Pharm. Res., 21, pp54-61, 1988) and 50 mg of Bifidobacterium KK-1 was added thereto and then was incubated at 37° C. for 72 hours. The incubates was centrifuged and the supernatant was concentrated and dried to obtain 580, 450 and 410 mg of processed extract of Panax ginseng root, Panax quinquefolia root and Panax notoginseng root respectively.

### Example 3

Preparation of Processed Extract of Panax Genus Plant

Each acid-treated extract prepared by Comparative Example 2 in an amount equivalent to 1 g of plant material, i.e., Panax ginseng root, Panax quinquefolia root and Panax notoginseng root was dissolved in 20 ml of distilled water. 50 mg of Bacterioides JY-6, 50 mg of Eubacterium L-8 and 50 mg of Bacteriodes stercoris was added thereto and then was incubated at 37° C. for 48 hours. The incubates was centrifuged and the supernatant was concentrated and dried to obtain 580, 630 and 450 mg of processed extract of Panax ginseng root, Panax quinquefolia root and Panax notoginseng root respectively.

### Example 4

Preparation of Processed Extract of Panax Genus Plant

Acid-treated extract prepared by Comparative Example 2 in an amount equivalent to 1 g of Panax ginseng root was dissolved in 20 ml of distilled water. 50 mg of Bifidobacterium K-506 (Disclosed in Arch. Pharm. Res., 21, pp54-61, 1988) and 50 mg of Bifidobacterium K-103 (Disclosed in Arch. Pharm. Res., 21, pp54-61, 1988) was added thereto and then was incubated at 37° C. for 72 hours. The incubates was centrifuged and the supernatant was concentrated and dried to obtain 430 mg of processed extract of Panax ginseng root.

### Example 5

Preparation of Processed Extract of Panax Genus Plant

Non-processed extract prepared by Comparative Example 1 in an amount equivalent to 1 g of Panax ginseng root was dissolved in 20 ml of distilled water containing 1% citric acid and incubated at 60° C. for 5 hours. The pH of cultivates was adjusted with NaOH or Calcium glucuronic acid to 6.8-7.0 and centrifuged to obtain its supernatant. 50 mg of Bifidobacterium K-506 and 50 mg of Bifidobacterium KK-2 (wet weight) was added thereto and then was incubated at 37° C. for 48 hours. The incubates was centrifuged and the supernatant was concentrated and dried to obtain 350 mg of processed extract of Panax ginseng root.

### Example 6

Preparation of Processed Extract of Panax Genus Plant

1 g of sliced Panax ginseng leaves was dissolved in 200 ml of MeOH, was refluxed for 3 hours and then the solvent was removed under reduced pressure. The remaining residue was suspended in distilled water and extracted with ether to remove ether soluble compounds. Remaining water layer was extracted with butanol and concentrated to obtain butanol soluble fraction. The butanol soluble fraction was heated at 130° C. for 3 hours and then 20 ml of distilled water was added to dissolve the solution. 100 mg of fresh human intestinal-bacterial colony was added thereto, and then was incubated at 37° C. for 48 hours. The incubates was centrifuged and the supernatant was extracted with 50 ml of butanol, concentrated in vacuo and dried to obtain 100 mg of processed extract of Panax ginseng leaves.

### Example 7

Preparation of Processed Extract of Panax Genus Plant

10 l of MeOH was added to 1 kg of dried 6 year old white ginseng, and extracted five times at room temperature for 48 hours, and concentrated in vacuo to obtain 50 g of methanol soluble extract (yield: 5%). 300 ml of distilled water was added thereto and then suspended to make its suspension solution. 500 ml of butanol was added thereto and then fractioned three times to obtain 15 g of saponin fraction.

1000 ml of distilled water containing 0.1% lactic acid was added to 15 g of above saponin fraction and incubated at 60° C. for 5 hours to obtain acid treated ginseng. The incubates were neutralized with NaOH and diluted with optimum amount of water. 15 g of Bifidobacterium KK-1 (No. of Subscription: KCCM 10364) and 15 g of Bifidobacterium KK-2 (No. of Subscription: KCCM 10365) was added thereto and then was incubated at 37° C. for 72 hours. The incubates were extracted with 1000 ml of butanol twice, concentrated in vacuo and dried to obtain 8.5 g of processed saponin fraction, the fraction was dissolved in distilled water and fresh human intestinal-bacterial colony was added thereto, and then was incubated at 37° C. for 48 hours. The incubates was centrifuged and the supernatant was extracted with 50 ml of saturated butanol, concentrated in vacuo and dried to obtain 100 mg of processed extract. 8.5 g of saponin fraction was subjected to silicagel column chromatography (3.5×60 cm, developing solvent: CHCl₃-MeOH=10:1) to give 100 mg of 20(S)-ginsenoside Rg₃, 50 mg of 20(R)-ginsenoside Rg₃, 10 mg of ginsenoside Rg₅, 10 mg of ginsenoside Rk₁, 70 mg of 20(S)-ginsenoside Rh₂, 8 mg of 20(R)-ginsenoside Rh₂, 15 mg of ginsenoside Rh₃, 10 mg of ginsenoside Rk₂, 10 mg of 20(S)-protopanaxadiol, 2 mg of 20(R)-protopanaxadiol, 5 mg of 20-dehydroprotopanaxadiol, 15 mg of ginsenoside Rh₁, 12 mg of protopanaxatriol.

### Experimental Example 1

Content Analysis Experiment

Each extract obtained from above Comparative Example 1, 2,3 and Example 1, 2, 3 in an amount equivalent to 500 mg of plant material was suspended with distilled water and extracted with n-BuOH. The butanol soluble layer was concentrated in vacuo and remaining residue was dissolved in 5 ml of MeOH. The solution was subjected to membrane filtration and injected to HPLC apparatus to determine the amount of saponin components therein.

The determination method of the amount of saponins therein was slightly modified with the methods disclosed in the literature (Kwon et al., J. Chromatography, A 921, pp335-339, 2001) and the determination condition of HPLC was as following:

Column: LiChrosorb RP-18

Elution solvent: A═H₂O, B═CH₃CN, A slope retention

O min (B 15%); 10 mins (B 34.5%); 5 mins (B 47.5%); 40 mins (B 80%); 50 mins (B 100%)

Flow rate: 1 ml/min.

Detector: Evaporated Light Scattering Detector (ELSD)

The results thus obtained are shown in Table 1 below

TABLE 1
The variation of relative amount of saponin component according to
processing method

| | Sample¹⁾ | Rk₂ + Rh₃ | PPD²⁾ | DHPPD³⁾ | Rg₃ | Rg₅ + Rk₁ | Ratio⁴⁾ |
|---|---|---|---|---|---|---|---|
| Comparative | A | — | — | — | — | — | — |
| Example 1 | B | — | — | — | — | — | — |
| | C | — | — | — | — | — | — |
| Comparative | A | — | — | — | 28 | 10 | — |
| Example 1 | B | — | — | — | 32 | 12 | — |
| | C | — | — | — | 25 | 8 | — |
| Comparative | A | — | — | — | 34 | 47 | — |
| Example 1 | B | — | — | — | 25 | 42 | — |
| | C | — | — | — | 22 | 35 | — |
| Example 1 | A | 14⁵⁾ | 5 | 1 | 13 | 20 | 0.61 |
| | B | 10 | 3 | 2 | 10 | 17 | 0.56 |
| | C | 9 | 2 | 3 | 8 | 12 | 0.70 |
| Example 2 | A | 12 | 8 | 3 | 11 | 20 | 0.74 |
| | B | 10 | 5 | 3 | 10 | 22 | 0.56 |
| | C | 7 | 3 | 2 | 6 | 14 | 0.60 |
| Example 3 | A | 2 | 2 | 1 | 13 | 5 | 0.28 |
| | B | 3 | 2 | 1 | 15 | 4 | 0.32 |
| | C | 2 | 1 | 1 | 14 | 3 | 0.23 |

¹⁾A: Panax ginseng B: Panax quinquefolia and C: Panax notoginseng
²⁾PPD: Protopanaxadiol
³⁾DHPPD: 20-Dehydroprotopanaxadiol
⁴⁾Ratio: (Rk₂ + Rh₃ + PPD + DHPPD)/(Rg₃ + Rg₅ + Rk₁)
⁵⁾Concentration denotes a ratio of the area of each saponin peak to that of total saponin

As a result, nonpolar saponin components such as ginsenoside Rg₃, Rg₅, Rk₁, Rk₂, Rh₃, PPD and DHPPD in the sample of Comparative Example 1, was not detected, which shows that non-processed ginseng itself do not contains those saponins. However, table 1 showed that the content of ginsenoside Rg₃ in the extract prepared by Comparative Example 2, are relatively higher than other components. In the extract prepared by Comparative Example 3, the amount of ginsenoside Rg₃, Rg₅ and Rk₁ are relatively higher than that of other components and ginsenoside Rk₂, Rh₃, PPD and DHPPD were not detected or merely detected. However, table 1 showed that the extract prepared by Example 1, 2 and 3, contained high amount of ginsenoside Rk₂, Rh₃, PPD and DHPPD.

### Experimental Example 2

Inhibitory Effect of Helicobacter pylori

In order to confirm the anti-helicobacter effect of the processed extract of Panax genus plant in the present invention, the experiment was performed by the procedure described in the literature (Bae, E.A. et al., Planta Med., 65, pp442-443, 1999).

Method

Six strains of Helicobacter pylori, i.e., ATCC 43504, NCTC 11637, NCTC 11638, Clinical 82516, Clinical 82548, Clinical 4 were inoculated to Brucella agar broth supplemented with 7% heat inactivated horse serum, cultivated at 37° C. for 3 days under anaerobic condition (5% O₂, 15% CO₂ and 80% N₂ gas) and 2 ml of physiologically saline solution was added to the plate in which each strain was grown to collect. 0.5 ml of above strain solution was transferred to 20 ml of Brucella broth medium containing 10% FBS(Fetal Bovine Serum), cultivated for 3 days under anaerobic condition (5% O₂, 15% CO₂ and 80% N₂ gas) and DMSO was added to be 10% solution, kept at −70° C. to use as a test strain. 0.7 ml of uniform concentration(10 mg/ml) of sample solution was added to 6.3 ml of to Brucella agar medium containing 7% heat inactivated horse serum, mixed to adjust final concentration of sample to 1 mg/ml and Helicobacter strain was transferred thereto, cultivated at 37° C. for 3 days under anaerobic condition (5% O₂, 15% CO₂ and 80% N₂ gas) and the proliferation rate of the strain was observed. Several samples i.e., the non-processed extract in Comparative Example 1, the acid treated extract in Comparative Example 2, heat treated extract in Comparative Example 3, processed extract in Example 1 to 3, the saponin fractions in Example 7 and the saponin compounds in Example 9, were added to Brucella agar medium and then the inhibition effect of proliferation of Helicobacter strain was observed, in particular, the inhibition result of compound showing potent activity was calculated with their MIC(minimum inhibition concentration) as shown in Table 2.

Result

As can be seen in Table 2, the processed extract in Example 1 to 3 shows most potent inhibiting activity of proliferation of Helicobacter strain among the test samples. 20(S)-protopanaxadiol shows potent inhibiting activity of proliferation of Helicobacter strain among the test saponin compounds and the value of MIC of panaxatriol and protopanaxadiol were 50 μg/ml respectively.

TABLE 2
inhibitory effect of proliferation of Helicobacter pylori (MIC, unit μg/ml)

| | | MIC (μg/ml) | | | | | |
|---|---|---|---|---|---|---|---|
| | | ATCC | NCTC | NCTC | Clinical | Clinical | Clinical |
| Sample | | 43504 | 11637 | 11638 | 82516 | 82548 | 4 |
| Com- | A¹⁾ | >1000 | >1000 | >1000 | >1000 | >1000 | >1000 |
| parative | B | >1000 | >1000 | >1000 | >1000 | >1000 | >1000 |
| Example 1 | C | >1000 | >1000 | >1000 | >1000 | >1000 | >1000 |
| Com- | A | 750 | 750 | 750 | 750 | 750 | 1000 |
| parative | B | 750 | 1000 | 750 | 750 | 750 | 1000 |
| Example 2 | C | 750 | 1000 | 1000 | 1000 | 750 | 1000 |
| Com- | A | 500 | 500 | 500 | 500 | 500 | 500 |
| parative | B | 500 | 500 | 500 | 500 | 500 | 500 |
| Example 3 | C | 500 | 500 | 500 | 750 | 500 | 500 |
| Example 1 | A | 125 | 125 | 125 | 125 | 125 | 125 |
| | B | 125 | 125 | 125 | 125 | 125 | 125 |
| | C | 125 | 125 | 125 | 125 | 250 | 125 |
| Example 2 | A | 125 | 125 | 250 | 250 | 125 | 250 |
| | B | 125 | 125 | 250 | 250 | 250 | 250 |
| | C | 250 | 250 | 250 | 250 | 250 | 250 |
| Example 3 | A | 250 | 125 | 250 | 250 | 250 | 250 |
| | B | 250 | 250 | 250 | 250 | 250 | 250 |
| | C | 250 | 250 | 250 | 250 | 250 | 250 |

| | | | | | | |
|---|---|---|---|---|---|---|
| Ginsenoside | >100 | >100 | >100 | >100 | >100 | >100 |
| Rb₁ | | | | | | |
| Ginsenoside | >100 | >100 | >100 | >100 | >100 | >100 |
| Rb₂ | | | | | | |
| Ginsenoside Rc | >100 | >100 | >100 | >100 | >100 | >100 |
| 20(S)-ginseno- | 200 | 200 | 200 | 200 | 200 | 200 |
| side Rg₃ | | | | | | |
| 20(R)-ginseno- | 200 | 200 | 200 | 200 | 200 | 200 |
| side Rg₃ | | | | | | |
| 20(S)-ginseno- | 100 | 100 | 100 | 100 | 100 | 100 |
| side Rh₂ | | | | | | |
| 20(R)-ginseno- | 100 | 100 | 100 | 100 | 100 | 100 |
| side Rh₂ | | | | | | |
| 20(S)-proto | 50 | 50 | 50 | 50 | 50 | 50 |
| panaxadiol | | | | | | |
| 20(R)-proto | 50 | 50 | 50 | 50 | 50 | 50 |
| panaxadiol | | | | | | |
| Compound K | >100 | >100 | >100 | >100 | >100 | >100 |
| Ginsenoside | >100 | >100 | >100 | >100 | >100 | >100 |
| Rh₁ | | | | | | |
| Protopanaxa- | 50 | 50 | 50 | 50 | 50 | 50 |
| triol | | | | | | |

¹⁾A: Panax ginseng B: Panax quinquefolia and C: Panax notoginseng

### Experimental Example 3

H⁺/K⁺-ATPase Activity Inhibition Test

In order to confirm the H⁺/K⁺-ATPase enzyme inhibitory activity of the extracts and the compounds isolated therefrom in the present invention, the experiment was performed by the procedure described in the literature (Bae, E.A. et al., Biol. Pharm. Bull., 25, pp58-63, 2002).

Method

Male Sprague-Dawley white mice weighed 200 g (Daehan Animals Co. Korea) were fasted for one night and anesthetized with ether. The stomach of mice was sliced, isolated and purified H⁺/K⁺-ATPase enzymes in stomach were isolated according to the method described in the literature (Sacoomani et al.; Biochem. Biophys. Acta, 912, pp63-73, 1987). 10 mM of imidazole buffer solution (pH 7.4) was added thereto and the solution was subjected to ultrasonication treatment (Ultrasonicater XL, Heat System Co.Ltd. USA), centrifuged at the speed of 1000 rpm for 30 minutes at 4° C. (Hanil HMR 210 IV High-speed centrifugal separator) and resulting supernatant was used as a test enzyme (Amount of the protein, 1 mg/ml). 0.5 ml of reaction mixture containing 0.1 ml of enzyme, 0.2 ml of 10 mM imidazole buffer solution(pH 7.4) and 0.2 ml of test sample, was pre-incubated at 37° C. for 30 minutes. Thereafter, reaction solution containing 4 mM of MgCl, 10 mM of ATP, 80 mM of imidazole buffer solution (pH 7.4) and 10 mM of KCl) was added thereto, reacted for 15 minutes and the reaction was quenched by the addition of 24% TCA (trichloroacetic acid). Phosphomolybdate-malachite green complex was added thereto to observe and determine their developed optical density (Van Veldhoven et al.; Anal. Biochem., 161, pp45-48, 1987).

Result

As can be seen in Table 3, the inhibition concentration for inhibiting H⁺/K⁺-ATPase enzyme by 50% (IC₅₀) of processed Panax plant extract in Example 1, 2 and 3 ranges 0.7 to 2.1 mg/ml and the inhibition concentration for inhibiting H⁺/K⁺-ATPase enzyme by 50% (IC₅₀) of 20(S)-ginsenoside Rh₂, 20(R)-ginsenoside Rh₂, 20(S)-ginsenoside Rg₃, 20(R)-ginsenoside Rg₃ were 0.5, 0.5, 0.6 and 0.7 mg/ml respectively. Omeprazole (ChongKeunDang Pharm. Co, Ltd. Korea) was used as a positive control.

TABLE 3
Inhibitory effect for H⁺/K⁺-ATPase (IC₅₀: unit mg/ml)

| Sample | | IC₅₀ (mg/ml) |
|---|---|---|
| Comparative Example 1 | A | >5 |
| | B | >5 |
| | C | >5 |
| Comparative Example 2 | A | 4.2 |
| | B | 4.6 |
| | C | 4.7 |
| Comparative Example 3 | A | 3.9 |
| | B | 4.1 |
| | C | 4.5 |
| Example 1 | A | 0.7 |
| | B | 0.8 |
| | C | 0.8 |
| Example 2 | A | 0.9 |
| | B | 1.1 |
| | C | 1.5 |
| Example 3 | A | 1.4 |
| | B | 1.8 |
| | C | 2.1 |

| | |
|---|---|
| Ginsenoside Rb₁ | >1 |
| Ginsenoside Rb₂ | >1 |
| Ginsenoside Rc | >1 |
| 20(S)-ginsenoside Rg₃ | 0.6 |
| 20(R)-ginsenoside Rg₃ | 0.7 |
| 20(S)-ginsenoside Rh₂ | 0.5 |
| 20(R)-ginsenoside Rh₂ | 0.5 |
| 20(S)-protopanaxadiol | >1 |
| Ginsenoside Rh₁ | >1 |
| Compound K | >1 |
| Omeprazole | 0.02 |

¹⁾A: Panax ginseng B: Panax quinquefolia and C: Panax notoginseng

As described above, it is confirmed that processed Panax genus plant prepared by the present invention shows more therapeutic and protective effect for gastro-intestinal diseases caused by abnormal proliferation of Helicobacter pylori than that of non-processed plant and thus, it is useful for anti-helicobacter drug or health care food.

### Experimental Example 5

Toxicity Test

Methods (1)

The acute toxicity tests on ICR mice (mean body weight 25±5 g) and Sprague-Dawley rats (235±10 g, Hyochang Science) were performed using the extract of the Example 1. Four group consisting of 10 mice or rats was administrated orally intraperitoneally with 500 mg/kg, 725 mg/kg, 1000 mg/kg and 5000 mg/kg of test sample or solvents (0.2 ml, i.p.), respectively, and observed for 2 weeks.

Methods (2)

The acute toxicity tests on ICR mice and Sprague-Dawley rats were performed using the extract of the Example 1. Four group consisting of 10 mice or rats was administrated intraperitoneally with 25 mg/kg, 250 mg/kg, 500 mg/kg and 725 mg/kg of test sample or solvents (0.2 ml, i.p.), respectively and observed for 24 hours.

Results

There were no treatment-related effects on mortality, clinical signs, body weight changes and gross findings in any group or either gender. These results suggested that the extract prepared in the present invention were potent and safe.

Hereinafter, the formulating methods and kinds of excipients will be described, but the present invention is not limited to them. The representative preparation examples were described as follows.

Preparation of Powder

| | |
|---|---|
| Dried powder of Example 1 | 50 mg |
| Lactose | 100 mg |
| Talc | 10 mg |

Powder preparation was prepared by mxing above components and filling sealed package.

Preparation of Tablet

| | |
|---|---|
| Ginsenoside Rh1 | 50 mg |
| Corn Starch | 100 mg |
| Lactose | 100 mg |
| Magnesium Stearate | 2 mg |

Tablet preparation was prepared by mixing above components and entabletting.

Preprartion of Capsule

| | |
|---|---|
| Dried powder of Example 1 | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of Injection

| | |
|---|---|
| Ginsenoside Rh1 | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of Liquid

| Dried powder of Example 1 | 0.1˜80 g |
|---|---|
| Sugar | 5˜10 g |
| Citric acid | 0.05˜0.3% |
| Caramel | 0.005˜0.02% |
| Vitamin C | 0.1˜1% |
| Distilled water | 79˜94% |
| CO₂ gas | 0.5˜0.82% |

Liquid preparation was prepared by dissolving active component, filling all the components and sterilizing by conventional liquid preparation method.

Preparation of Health Care Food

| Extract of Example 1 | 1000 | mg |
|---|---|---|
| Vitamin mixture | optimum amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above mentioned vitamin and mineral mixture may be varied in may ways. Such variations are not to be regarded as a departure from the spirit and scope of the present invention.

Preparation ot health beverage

| | | |
|---|---|---|
| Extract of Example 1 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A pharmaceutical composition comprising processed Panax plant or the extract thereof wherein the ratio of ginsenoside (Rk₂+Rh₃+protopanaxadiol+20-dehydroprotopanaxadiol) to (Rg₃+Rg₅+Rk₁) of above 0.1, as an active ingredient in an amount effective to treat or prevent human or mammal gastro-intestinal diseases caused by abnormal proliferation of Helicobacter pylori, together with a pharmaceutically acceptable carrier.

2. The pharmaceutical composition according to claim 1, wherein said ratio is above 0.2.

3. The pharmaceutical composition according to claim 1, wherein said ratio is above 0.5.

4. A pharmaceutical composition comprising the extract of Panax genus plant obtained by the steps essentially comprising acid or heat treating or their combination thereof the plant material belonged to Panax genus and subsequent fermentation treating with lactic-acid bacteria or intestinal bacteria, as an active ingredient in an amount effective to treat or prevent human or mammal gastro-intestinal diseases caused by abnormal proliferation of Helicobacter pylori, together with a pharmaceutically acceptable carrier.

5. The pharmaceutical composition according to claim 4, wherein said Panax genus plant comprises at least one selected from the group consisting of Panax ginseng, Panax quinquefolia, Panax notoginseng, Panaxjaponica, Panax trifolia, Panax pseudoginseng, Panax vietnamensis, Panax elegatior, Panax wangianus, Panax bipinratifidus and Panax angustifolium.

6. The pharmaceutical composition according to claim 4, wherein said plant material comprises the root, stem, petal, leaf, fruit and their tissue cultivates thereof.

7. The pharmaceutical composition according to claim 4, wherein said plant material comprises fresh ginseng, processed ginseng or ginseng by-product thereof.

8. The pharmaceutical composition according to any of claim 1 to 7, wherein said gastro-intestinal disease comprises gastritis, gastric ulcer, duodenal ulcer and gastric cancer.

9. A pharmaceutical compositions comprising saponin compounds selected from the group consisting of panaxytriol, panaxydol, panaxynol, ginsenoside Rc, Rb₁, 20(S)-ginsenoside Rg₃, 20(R)-ginsenoside Rg₃, 20(S)-ginsenoside Rh₂, 20(R)-ginsenoside Rh₂, 20(R)-protopanaxadiol, 20(S)-protopanaxadiol, 20(S)-ginsenoside Rh₁, 20(S)-protopanaxatriol and the mixture thereof, as an active ingredient in an amount effective to treat or prevent human or mammal gastro-intestinal diseases caused by abnormal proliferation of Helicobacter pylori, together with a pharmaceutically acceptable carrier.

10. The pharmaceutical composition according to any of claims 1 to 9, wherein said pharmaceutical composition is provided in an acceptable carrier as powder, granule, tablet, capsule, aqueous medicine or injection.

11. A method for treating or preventing gastro-intestinal disease in a mammal comprising administrating to said mammal an effective amount of the extract wherein the ratio of ginsenoside (Rk₂+Rh₃+protopanaxadiol+20-dehydroprotopanaxadiol) to (Rg₃+Rg₅+Rk₁) of above 0.1, together with a pharmaceutically acceptable carrier thereof.

12. A method for treating or preventing gastro-intestinal disease in a mammal comprising administrating to said mammal an effective amount of Panax genus plant or the extract thereof obtained by the steps essentially comprising acid or heat treating or their combination thereof the plant material belonged to Panx genus and subsequent fermentation treating with lactic-acid bacteria or intestinal bacteria, together with a pharmaceutically acceptable carrier thereof.

13. A method for treating or preventing gastro-intestinal disease in a mammal comprising administrating to said mammal an effective amount of a compound selected from the group consisting of panaxytriol, panaxydol, panaxynol, ginsenoside Rc, Rb₁, 20(S)-ginsenoside Rg₃, 20(R)-ginsenoside Rg₃, 20(S)-ginsenoside Rh₂, 20(R)-ginsenoside Rh₂, 20(R)-protopanaxadiol, 20(S)-protopanaxadiol, 20(S)-ginsenoside Rh₁, 20(S)-protopanaxatriol and the mixture thereof, together with a pharmaceutically acceptable carrier thereof.
